(19) United States
(12) Patent Application Publication
(10) Pub. No.: US 2017/0347917 A1
(43) Pub. Date: Dec. 7, 2017

(54) NEWBORN RESPIRATION MONITORING SYSTEM AND METHOD

(71) Applicant: General Electric Company, Schenectady, NY (US)

(72) Inventors: Steven Mitchell Falk, Baltimore, MD (US); Karen P. Starr, Monkton, MD (US); Sri Ramaprasad Prasad, Ellicott City, MD (US)

(73) Assignee: General Electric Company, Schenectady, NY (US)

(21) Appl. No.: 15/174,158

(22) Filed: Jun. 6, 2016

(51) Int. Cl. A61B 5/087; A61B 5/08; A61B 5/145; A61M 16/08; A61B 5/00; A61M 16/06; A61B 5/091; A61M 16/00

(52) U.S. Cl. CPC A61B 5/087; A61M 16/06; A61B 5/0816; A61B 5/082; A61B 5/091; A61B 5/14542; A61M 16/0875; A61B 5/0022; A61B 5/742; A61B 5/7275; A61M 2016/0033; A61M 2205/3331; A61M 2205/3368; A61M 2230/432; A61M 2230/435

(57) ABSTRACT

A newborn respiration monitoring system includes a flow sensor that measures a gas flow and a CO₂ sensor that measures a CO₂ within the breathing circuit for an infant. The system further includes a resuscitation module executable on a processor of a computing system to receive the flow measurement and the CO₂ measurement and determine respiratory information for the infant. A digital display is communicatively connected to the computing system and displays the respiratory information.

### BACKGROUND

The present disclosure relates to the field of newborn care, and more specifically to systems and methods for providing respiratory care to newborn infants immediately upon birth.

At the time of birth, infants need immediate assessment and care, including assessment of heart and respiratory function. Infant patients can experience relatively rapid changes in condition, especially immediately after birth. Depending on the infant's condition, various therapies may be provided, including resuscitation or other respiratory care.

### SUMMARY

This Summary is provided to introduce a selection of concepts that are further described below in the Detailed Description. This Summary is not intended to identify key or essential features of the claimed subject matter, nor is it intended to be used as an aid in limiting the scope of the claimed subject matter.

In one embodiment, a newborn respiration monitoring system includes a flow sensor that measures a gas flow and a CO₂ sensor that measures a CO₂ within the breathing circuit for an infant. The system further includes a resuscitation module executable on a processor of a computing system to receive the flow measurement and the CO₂ measurement and determine respiratory information for the infant. A digital display is communicatively connected to the computing system and displays the respiratory information.

One embodiment of a method of monitoring newborn infant respiration includes measuring a gas flow with a flow sensor in a breathing circuit for the infant, communicating the flow measurement to a computing system, measuring a CO₂ with a CO₂ sensor in a breathing circuit for the infant, and communicating the CO₂ measurement to the computing system. The method further includes determining respiratory information for the infant with the computing system based on at least the flow measurement and the CO₂ measurement, and displaying the respiratory information for the infant on a digital display communicatively connected to the computing system.

Various other features, objects, and advantages of the invention will be made apparent from the following description taken together with the drawings.

### BRIEF DESCRIPTION OF THE DRAWINGS

The present disclosure is described with reference to the following Figures.

FIG. 1 depicts one embodiment of a newborn respiration monitoring system incorporated in a mobile newborn care bed.

FIG. 2 is a schematic depicting one embodiment of a newborn respiration monitoring system.

FIG. 3 is a schematic depicting one embodiment of a computing system for a newborn respiration monitoring system.

FIG. 4 is a schematic depicting another embodiment of a newborn respiration monitoring system.

FIG. 5 is a flowchart depicting one embodiment of a method of monitoring newborn infant respiration.

FIG. 6 depicts another embodiment of a method of monitoring newborn infant respiration.

### DETAILED DESCRIPTION

In light of their experimentation and research in the relevant field, the present inventors have recognized that clinicians providing care to infants at birth are often seeking more guidance for providing safe, non-invasive respiratory and resuscitative care to infants, such as to reduce barotrauma and volutrauma, and to reduce or delay use of invasive ventilation as much as possible in the delivery room. Current systems for providing newborn resuscitation do not enable sufficient respiratory monitoring necessary to provide consistent and optimal resuscitative care to a newborn, including failing to provide barometric, volumetric, and inspiratory/expiratory gas content measurements. In light of these problems and needs in the relevant field recognized by the inventors, they developed the disclosed newborn respiration monitoring system and method, including sensor systems for monitoring non-invasive respiratory therapy providing positive pressure ventilation and/or continuous positive airway pressure.

Further, according to long-standing care standards, an infant's umbilical cord is cut immediately upon delivery and the infant was placed on a patient care surface spaced away from the mother to assess and provide any needed therapy, such as respiratory support. In such instances, babies were removed from the delivery location and placed on a bassinet or infant bed, often containing a radiant warmer. Currently available infant beds and radiant warmers are configured to be positioned in a corner of a delivery room so as not to crowd the space next to the mother. Moreover, most infant care beds and radiant warmers are one, integrated, bulky device, where the bassinette is built into the warmer. Resuscitation equipment and/or monitoring equipment, if any, is either integrated into the warming device or positioned near the infant bed/radiant warmer away from the delivery location.

However, care standards are trending towards maintaining the infant at the birthing site to the extent possible in order to allow delayed cord clamping and delayed cutting for several minutes so that the blood in the placenta is transferred to the baby. Accordingly, through their experimentation and research in the relevant field, the present inventors have recognized such delated cord clamping and other modern care standards for newborn infants immediately after birth have made current radiant warmer and resuscitation platform technology challenging. The inventors have recognized that a device is needed to provide diagnosis and therapy to a newborn infant immediately next to the mother and at the site of birth so that such therapy can be administered before and/or during the cord clamping. Further, the inventors have recognized that devices and systems are needed that provide monitoring and resuscitation care for infants easily and with minimal attachment of devices to the baby. Further, the inventors have recognized that devices and systems are needed that provide immediate and accessible display of multiple relevant respiration-related parameters to clinicians providing care, and also seamless transmission and storage and of such data to the patient's healthcare records.

In view of their recognition of problems and needs in the relevant field, the inventors developed the disclosed newborn respiration monitoring system and method. The newborn respiration monitoring system is mobile and able to be located at a delivery location of an infant to enable a clinician to provide respiration monitoring and/or resuscitative care to an infant immediately upon birth at the birthing location, including before and during cord clamp.

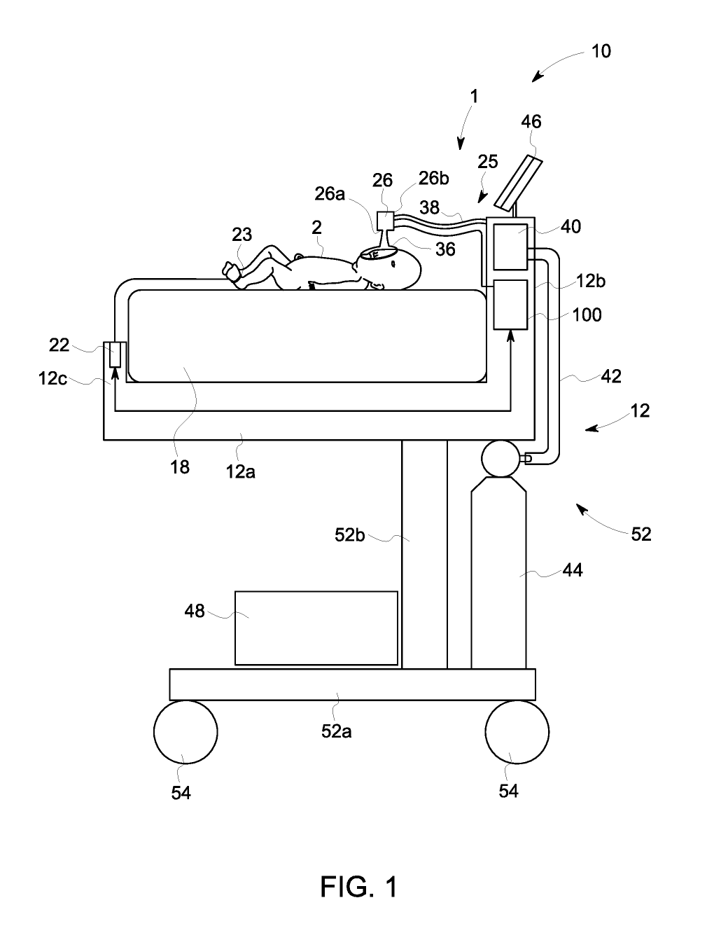

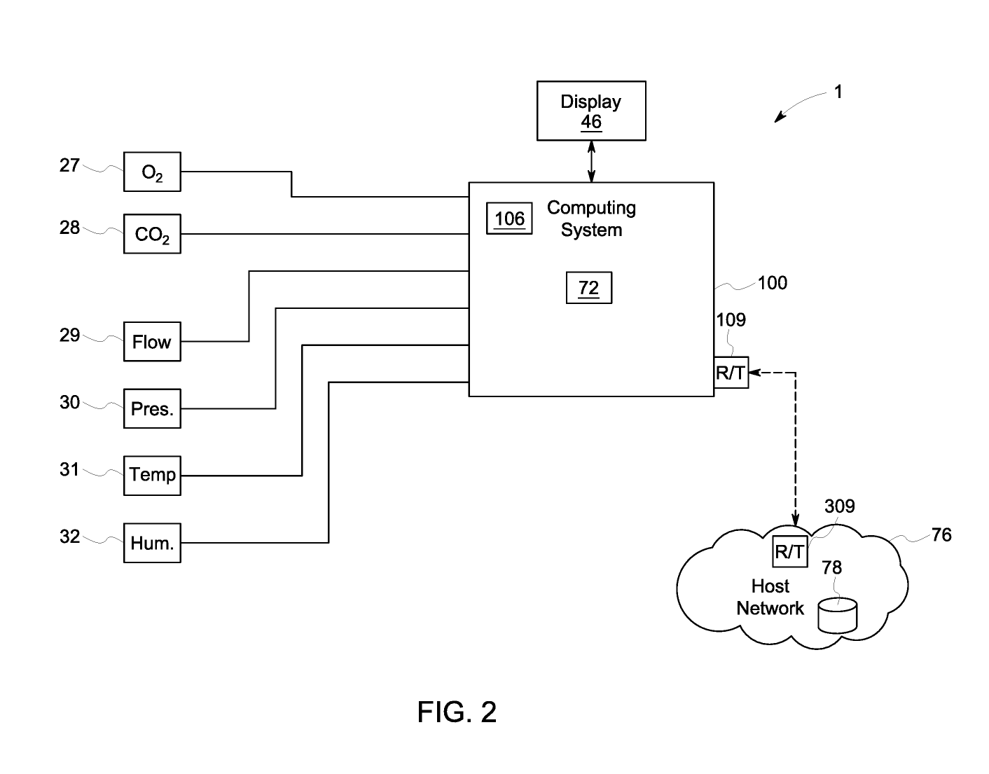

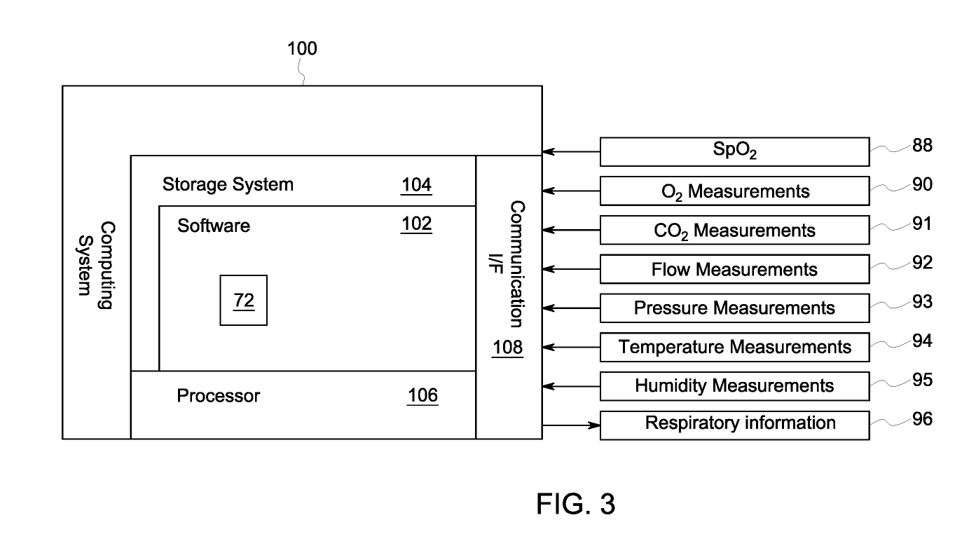

FIG. 1 depicts one embodiment of a newborn respiration monitoring system 1, which in the depicted embodiment is incorporated into a bassinette of a mobile newborn bed. FIGS. 2 and 3 provide schematic diagrams of various embodiments of the newborn respiration monitoring system 1, which may be a relatively small and portable system separate and apart from an infant bed and able to be transported to a delivery location of an infant. The newborn respiration monitoring system 1 includes one or more sensors to measure parameters within a breathing circuit 25 for the infant 2. Examples of the one or more sensors include an O₂ sensor 27, a CO₂ sensor 28, a flow sensor 29, a pressure sensor 30, a temperature sensor 31, and a humidity sensor 32. Each of the sensors measures a value within the breathing circuit 25 and communicates the value to a computing system 100. The computing system 100 includes a resuscitation module 72 executable on one or more processors 106 to determine respiratory information 96 for the infant 2. The computing system 100 may control a digital display 46 to display some or all of the respiratory information 96, such as to provide information to a clinician caring for the infant 2 regarding the infant's respiratory health and/or regarding the respiratory intervention being provided to the infant 2 via the breathing circuit 25. Additionally, the computing system 100 may communicate the respiratory information to a host network 76 and/or to an intermediary, such as hub device 68.

The newborn respiration monitoring system 1 may be a stand-alone system or set of devices that transportable to a location where respiratory intervention is being provided to an infant 2 with a respiratory device 40. Alternatively, the newborn respiration monitoring system 1 may be incorporated into another device for providing infant care, such as into a respirator device, a fetal monitor, or another device for monitoring the physiological well being of a newborn infant 2. FIG. 1 exemplifies an embodiment where the newborn respiration monitoring system 1 is incorporated into a bassinette 12 of a mobile newborn bed 10. The newborn bed 10 is preferably portable and small enough and agile enough to be transported to and located at the delivery location of the infant so that respiratory care and respiration monitoring can be provided to the infant 2 at the delivery location where the infant is delivered by the mother.

The mobile newborn bed 10 has a bassinet 12 and a frame 53. The bassinet contains a mattress 18 on which the infant 2 is placed. The mattress 18 is preferably a flat or slightly concave cushioned surface, but can be any flat or curved surface capable of receiving the infant 2. The frame 52 is underneath the bassinet 12 and supports the bassinet 12. The frame includes a base frame portion 52a connecting to one or more wheels 54 that allow the mobile newborn bed 10 to be easily moved. The frame 52 also includes a vertical frame portion 52b that elevates and attaches to the bassinet 12. In various embodiments, the vertical frame portion 52b may be adjustable to adjust the height of the bassinet 12. The base frame portion 52a may be configured to support various elements comprising part of the mobile newborn bed 10, such as one or more batteries 48 and/or gas supply tanks 44.

In the depicted embodiment, the bassinet 12 includes a bottom portion 12a supporting the mattress 18, and also includes a head portion 12b adjacent to one side of the mattress 18 and a foot portion 12c adjacent to another side of the mattress 18. In the depicted embodiment, the head portion 12b houses or comprises computing system 100 and respirator 40, and the foot portion 12c houses or comprises pulse oximeter device 22. In other embodiments, such devices may be housed or incorporated at other locations on the mobile newborn bed 10 or may be provided separately but in conjunction with the mobile newborn bed 10.

Devices and systems for providing resuscitation and other respiratory therapy to an infant 2 may be associated with or incorporated into the newborn respiration monitoring system 1, which includes sensors placed within a breathing circuit 25. A breathing circuit 25 for providing gas to the infant 2 may include a ventilator device 40, such as a continuous positive airway pressure (CPAP) device, a positive pressure ventilation (PPV) device, or a positive end-expiratory pressure (PEEP) device (or a ventilator device providing all three respiratory therapies). In the embodiment depicted in FIG. 1, the ventilator device 40 receives a gas supply from supply tube 42 connected to gas supply tank 44 supported on the base frame portion 52a. The ventilator device 40 regulates the gas supply as appropriate to provide resuscitative or respiratory assistance to the infant 2. The ventilator device 40 connects to the breathing tube 38 to supply gas to the infant through mask 36 applied over the infant's nose and mouth. In other embodiments, the breathing tube 38 may deliver gas to the infant 2 via a nasal cannula or by some other delivery means.

The breathing circuit 25 is equipped with sensors for measuring parameters relevant to the infant's respiration, which may be provided in the mask 36, breathing tube 38, or at the connection of the mask 36 and the breathing tube 38. Various sensors may be incorporated into the breathing circuit 25, such as a CO₂ sensor 28 that measures CO₂ in gas expired by the infant 2, an O₂ sensor 27 that measures O₂ in gas inspired by the infant 2, a flow sensor 29 that measures gas flow at a location in the breathing circuit 25, a pressure sensor 30 that measures pressure at a location in the breathing circuit 25, a temperature sensor 31 measuring temperature of expired and/or inspired gas within the breathing circuit 25, and/or a humidity sensor 32 measuring humidity of inspired gas within the breathing circuit 25. More specifically, the O₂ sensor 27 supplies O₂ measurements 90, CO₂ sensor 28 supplies CO₂ measurements 91, flow sensor 29 supplies flow measurements 92, pressure sensor 30 supplies pressure measurements 93, temperature sensor 31 supplies temperature measurements 94, and humidity sensor 32 supplies humidity measurements 95.

As shown in FIG. 1, the mobile newborn bed 10 may include a battery 48 to power the various devices thereon, including some or all of the various sensing devices, the computing system 100, the ventilator device 40, and/or the digital display 46. The battery 48 may be positioned on the base frame portion 52a, for example, and in such a location to be easily accessed in order to recharge or replace the battery 48. The charge status of the battery 48 may be monitored by a power control module, such as may be provided separately from and in communication with, or otherwise incorporated into, the computing system 100. Further, the computing system 100 may provide a battery status notification, such as on digital display 46, regarding the charge of the battery 48 on the digital display 46 so that a clinician or other user will be able to determine the charge level of the battery 48.

In various embodiments, newborn respiration monitoring system 1 may be configured with any one or more of the aforementioned sensors to provide respiration parameter measurements 90-95 from the breathing circuit 25, and such respiration parameter measurements may include, but are not limited to, the aforementioned measurements. The respiration parameter measurements 90-95 are communicated to computing system 100 by wired or wireless means. For example, each of the sensors 27-32 may be incorporated into the patient-end of the breathing circuit 25, such as in the mask 36, breathing tube 38, or at a junction therebetween, and such sensors may connect by wires running along the breathing tube 38. In some embodiments such wires may be incorporated into the length of the breathing tube 38. In other embodiments, one or more of the sensors 27-32 may be equipped with or associated with a wireless transmitter to wirelessly transmit the respiration parameter measurements 90-95 to the computing system 100, and in such embodiments may also be associated with or include an analog-to-digital converter to digitize analog signals before wireless transmission.

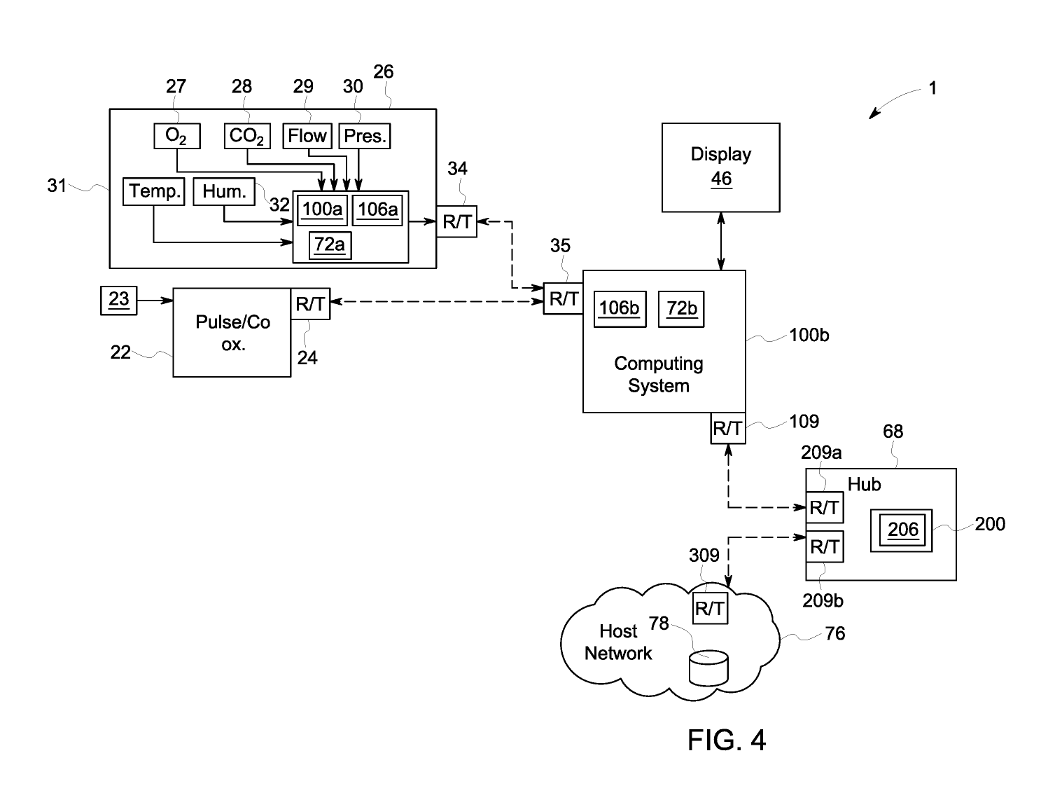

For example, each of the aforementioned sensors 27-32 may be contained in a respiration sensor device 26 positioned in the breathing circuit 25, such as between the mask 36 and the breathing tube 38. FIG. 4 schematically depicts an exemplary embodiment of the respiration sensor device 26 containing O₂ sensor 27, CO₂ sensor 28, flow sensor 29, pressure sensor 30, temperature sensor 31, and humidity sensor 32. For instance, the respiration sensor device 26 may be configured to communicate the respiration parameter measurements 90-95 from all of the sensors 27-32 to the computing system 100. The respiration sensor device 26 may communicate wirelessly or by wires that extend to the computing system 100. In the embodiment of FIG. 1, wires (such as extending along and/or embedded into the breathing tube 38) connect one or more sensors 27-32 to a receiving connector in the bassinet 12, or otherwise electrically connect to the computing system 100. In other embodiments, such as that depicted in FIG. 4, the respiration sensor device 26 may communicate the respiration parameter measurements 90-95 to a wireless receiver associated with the computing system 100.

The computing system 100 may be communicatively connected (i.e. connected by physical or wireless means so as to be able to communicate messages to or with another device) to digital display 46 to communicate display commands thereto, such as to display the respiratory information 96 thereon. Accordingly, the digital display 46 may display the infant's respiration rate, FiO₂, etCO₂, or any of numerous other respiratory information 96 to a clinician while the clinician is providing medical care to the infant 2. Likewise, the computing system 100 may control the digital display 46 to display notifications of inappropriate respiratory intervention, poor respiratory health or respiratory events, such as to provide a visual alert when one or more values in the respiratory information 96 is outside of a predetermined range or changes by more than a predetermined amount over a short period of time.

The digital display 46 may be any digital display device known in the art and may be a housed separately from the computing system 100 or housed together with the computing system 100. In the context of the FIG. 1 embodiment, the digital display may be fixed to the bassinet 12, such as to the head portion 12b of the bassinet 12, in a way that is visible to clinicians providing care to the infant 2. Alternatively, the digital display 46 may be a separable or completely separate device from the bassinette 12, such as a tablet or mobile computer. In still other embodiments, the digital display 46 may be a display of another device networked with the computing system 100 of the newborn respiration monitoring system 1, such as a display of a fetal monitor. Likewise, in an embodiment where the newborn respiration monitoring system is incorporated into or with another monitoring device, the computing system 100 may be a shared computing system with multiple monitoring functions.

In an embodiment where the newborn respiration monitoring system 1 is a stand-alone device, the computing system 100 may be housed separately from or together with the digital display 46 and the sensors 27-32. For example, the computing system 100 may be incorporated into the same housing as the digital display 46, or it may be partially or entirely incorporated into a housing with one or more of the sensors 27-32. FIG. 4 exemplifies one embodiment where the computing system 100 comprises a first computing system portion 100a incorporated into respiration sensor device 26 and a second computing system portion 100b communicatively connected to digital display 46. As described further herein, the various functions of the computing system 100 and resuscitation module 72 may be divided between multiple locations and executed on different processors.

The newborn respiration monitoring system 1 may further include a pulse oximeter device 22, including sensor 23 attachable to the patient that determines an estimate of oxygen saturation (SpO₂) value 88 and transmits the SPO₂ value 88 to the computing system 100. The pulse oximeter 22 may transmit the SpO₂ value by wired or wireless means, various examples of which are provided herein. In an embodiment like that of FIG. 1 where the newborn respiration monitoring system 1 is incorporated into a mobile newborn bed 10, the pulse oximeter 22 may be incorporated into the bassinet 12, such as in the foot portion 12c. In other embodiments, the pulse oximeter may be a separate device that may be kept in proximity of the bassinet 12 and may be wirelessly paired with the computing system 100. As exemplified in the embodiment of FIG. 4, the pulse oximeter 22 is provided with receiver/transmitter 24, which communicates with receiver/transmitter 35 of the second computing system portion 100b.

The sensor 23 may be any sensor device capable of measuring the infant's peripheral oxygen saturation or other hemoglobin saturation parameters, such as a disposable adhesive sensor device configured to wrap around the infant's foot. The sensor 23 may include a wire connecting to the pulse oximeter 22. In still other embodiments, the physical circuitry and software of the pulse oximeter 22 may be incorporated within the computing system 100, and thus the sensor 23 may communicate measurements related to O₂ saturation directly to the computing system 100 for determination of SpO₂ values 88 for the infant 2.

Upon receipt or determination of the SpO₂ value 88 for the infant 2, the computing system 100 may transmit the SpO₂ value 88 to the hub device 68, or directly to a host network 76. Further, the computing system 100 may send control signals to the digital display 46 in order to display the SpO₂ value 88 thereon. Alternatively or additionally, the device 22 may be a co-oximeter device that measures and determines one or more of SpO2, carboxyhemoglobin saturation (SpCO), methemoglobin saturation (SpMet), and/or total hemoglobin concentration (g/dl SpHb). For instance, the co-oximeter device 22 may be a Rainbow SET Pulse CO-Oximeter by Masimo Corporation of Irvine, Calif. The SpO₂, SpCO, SpMet and/or SpHb can relate to respiration and can provide useful information regarding what and how respiratory intervention should be applied. Accordingly, the newborn respiration monitoring system 1 may incorporate such measurements in its overall display of information to a clinician providing care for the infant 2, so that the infant's condition can be immediately assessed and it can be determined what resuscitative care is necessary and appropriate.

As described herein, the digital display 46 may be controlled by the computing system 100 to provide various health information for the patient, including the respiratory information 96, SPO₂ value 88, or any other relevant value. Additionally, the digital display 46 may provide a user input device, such as via a touchscreen, to provide control input to the computing system 100 and/or any other system or device incorporated in or associated with the newborn respiration monitoring system 1. Accordingly, in various embodiments, multiple systems and devices may connect directly to the digital display 46 and be capable of providing control signals to the digital display 46. For example, the ventilator device 40 may connect to the digital display 46 and the digital display 46 may provide a user interface to control the ventilator device 40. Such connectivity may be provided directly between the ventilator device 40 and the digital display 46, or may be routed through the computing system 100, which may provide a central control for multiple devices, such as including the ventilator device 40.

Referring to FIGS. 2 and 3, the computing system 100 may include a software module stored in memory and executable on a processor 106 within the computing system 100, a resuscitation module 72, configured to process one or more of the respiration parameter measurements 90-95 to generate respiratory information 96 regarding the respiratory status of the infant 2. For example, the resuscitation module 72 may determine respiratory information 96 including an inspired O₂ indicator, such as fraction of inspired oxygen (FiO₂). Alternatively or additionally, respiratory information 96 determined by the resuscitation module 72 may include an end tidal CO₂ (etCO₂) based on the CO₂ measurements 91. Likewise, resuscitation module 72 may calculate tidal volume based on the flow measurements 92, such as by calculating volume as an integral of the flow curve and/or sum of the flow measurements 92 during the inspiratory cycle, and/or intake air pressure based on the pressure measurements 93. Alternatively or additionally, the resuscitation module 72 may utilize the temperature measurements 94 to determine the temperature of the inspired gas and/or the expired gas. Such temperature measurements 94 may be used to regulate the temperature of the gas provided to the infant 2 and/or to determine information about the temperature of the infant 2. The resuscitation module 72 may utilize the humidity measurements 95 to determine a humidity of the gas being provided to the patient, and such information may be used to control the same. Any one of the aforementioned values may be included in the respiratory information 96, which may also include any number of alternative or additional parameters (e.g., respiration rate) outputted by the resuscitation module 72, and such respiratory information 96 may be transmitted to a hub device 68 and/or a host network 76 for storage in the patient's medical record in database 78. Alternatively or additionally, some or all of the respiratory information 96 may be displayed on the digital display 46.

FIG. 4 schematically depicts an exemplary embodiment of the newborn respiration monitoring system 1 that includes a respiration sensor device 26 containing O₂ sensor 27, CO₂ sensor 28, flow sensor 29, pressure sensor 30, temperature sensor 31, and humidity sensor 32. The respiration sensor device 26 further includes a first computing system portion 100a having processor 106a and first resuscitation module portion 72a executed on processor 106a receives the respiration parameter measurements 90-95 from the sensors 27-31. A person having ordinary skill in the art will understand in light of this disclosure that computing system portions 106a and 106b may be independent computing systems communicatively connected as part of the newborn respiration monitoring system 1 and to execute the methods 140 described herein. Likewise, a person having ordinary skill in the art will understand in light of this disclosure that the computing system portions 100a, 100b may be housed in any of various components within the system 1, such as in the respirator device 40 or incorporated as part of another fetal monitor or fetal care device or system. The first computing system portion 100a and resuscitation module portion 72a may filter and condition the signals for transmission to the second computing system portion 100b and second resuscitation module portion 72b. A person having ordinary skill in the art will understand in light of this disclosure that such sensors 27-32 may be analog or digital, producing analog or digital respiration parameter measurements 90-95, and thus analog-to-digital conversion circuitry may be incorporated in the respiration sensor device 26 as necessary to digitize measurements from analog sensor devices. The first resuscitation module portion 72a may process some or all of the respiration parameter measurements 90-95 to respiratory information 96 for the infant.

The first computing system portion 100a and first resuscitation module portion 72a communicates the respiration parameter measurements 90-95 and/or respiratory information 96 via wireless communication protocol to second computing system portion 100b through wireless receiver/transmitter 34. Transmissions from the wireless receiver/transmitter 34 are received by a wireless receiver/transmitter 35 associated with the computing system 100. The wireless receiver/transmitters 34 and 35 may communicate via any wireless protocol, and relatively short range wireless protocols, such as Bluetooth, Bluetooth low energy (BLE), ANT, ZigBee, or a near field communication (NFC) protocol, may be especially useful in embodiments of the newborn respiration monitoring system 1 where the distance between the respiration sensor device 26 and the second computing system portion 100b are expected to be small. In other embodiments, the communication may be via network protocols appropriate for longer-range wireless transmissions, such as on the wireless medical telemetry service (WMTS) spectrum or on a Wi-Fi-compliant wireless local area network (WLAN). In still other embodiments, the receiver/transmitters 109 and 209a may be capable of switching between two or more wireless communication protocols, such as to optimize data communication based on the situation.

The respiration sensor device 26 may be configured to be positionable between the mask 36 and the breathing tube 38. Referring to FIG. 1, the respiration sensor device 26 may have a first end 26a connectable to mask 36 (or other gas delivery means, such as nasal prongs) and a second end 26b connectable to breathing tube 38. Accordingly, each end 26a, 26b may have appropriate connecting means to facilitate such connection within the breathing circuit. Furthermore, the first end 26a and second end 26b may be configured in any position with respect to one another on the respiration sensor device, and may be positioned oppositely, perpendicularly, or adjacently to one another on the respiration sensor device 26. In another embodiment, the respiration sensor device 26 may be incorporated into the mask 36, such that the respiration sensor device 26 is a single, inseparable element with the mask 36.

In FIG. 4, the second computing system portion 100b and the second resuscitation module portion 72b receive the respiration parameter measurements 90-95 and/or respiratory information 96 and conduct further processing as required to generate further respiratory information 96 and/or conduct further assessment of the data. For example, the second resuscitation module portion 72b may determine one or more respiratory information trends, such as by plotting some or all of the respiratory information 96 with respect to time. The second resuscitation module portion 72b may further control the digital display 46 to display some or all of the respiratory information 96 or respiratory information trends. The second computing system portion 100b communicates wirelessly to a hub device 68, which in turn communicates wirelessly to host network 76. The hub device 68 may be may be positioned at any location within communication distance of the second computing system portion 100b. The hub device 68 may be provided by a mobile computing device, such as a laptop, tablet, smart phone, or the like. For example, a software application may be provided to allow a clinician's tablet or smart phone to act as the hub device 68. In still other embodiments, the hub device 68 may be a fetal monitoring unit, and thus the second computing system portion 100b may communicate the respiratory information 96 and or respiration parameter measurements 90-95 to the fetal monitoring unit for transmission to the host network 76. In such an embodiment, the fetal monitoring unit may also provide the digital display 46 to display some or all of the respiratory information 96, etc.

In an embodiment incorporating a hub device 68, the hub device 68 has a computing system 200 equipped with a processor 206. The hub computing system 200 is equipped to communicate with the computing system 100 and the host network 76 via receiver/transmitters 209a and 209b respectively. Wireless communication between the hub device 68 and the host network 76, or between the computing system 100 and the host network 76, may accomplished by any wireless protocols known in the relevant art. In the depicted embodiments, the computing system 100 has receiver/transmitter 109 configured to communicate with receiver/transmitter 209a on the hub device 68. The various receiver/transmitters 24, 34, 35, 109, 209a, 209b, 309 may include separate receiving and transmitting devices or may include an integrated device providing both functions, such as a transceiver. The computing system 100 and hub device 68, via respective receiver/transmitters 109 and 209a, may be configured as medical body area network (MBAN) devices. In other embodiments, the receiver/transmitters 109 and 209a, and/or 209b and 309 may communicate via other short range radio protocols, such as Bluetooth, Bluetooth Low Energy (BLE), ANT, ZigBee, or NFC. In other embodiments, the communication may be via network protocols appropriate for longer-range wireless transmissions, such as on the wireless medical telemetry service (WMTS) spectrum or on a Wi-Fi-compliant wireless local area network (WLAN). In still other embodiments, the respective receiver/transmitters may be capable of switching between two or more wireless communication protocols, such as to optimize data communication based on the situation.

In other embodiments, where the computing system 100 communicates directly with the host network 76 via communication between receiver/transmitters 109 and 209, such transmission may be via network protocol appropriate for longer-range wireless transmissions, such as on the WMTS spectrum or on a WLAN, as described above. The host network 76 may be, for example, a local computer network having servers housed within a medical facility where the infant 2 is born, or it may be a cloud-based system housed by a cloud computing provider. The host network 76 may include a medical records database 78 housing the medical records for the infant 2, which may be updated to store the information transmitted by the computing system 100 and/or the hub device 68.

FIG. 3 provides a system diagram of a computing system 100 having resuscitation module 72 executable to determine respiratory information 96. Furthermore, the resuscitation module 72 executable to store the respiratory information 96 in storage system 104 of the computing system 100 so that such information may be accessed at a later time, such as to generate trend plots. Likewise, resuscitation module 72 may be executable to store the measurement data from the sensors 27-32, in storage system 104 of the computing system 100 so that such information may be accessed at a later time, such as to generate trend plots. For example, such information may be accessed by the various modules and/or by clinicians to determine whether the infant 2 is ready for discharge or whether certain physiological indicators indicate that continued care is needed, such as whether the infant 2 is experiencing continued apnea events.

Computing system 100 includes a processor 106, storage system 104, software 102, and communication interface 108. The processor 106 loads and executes software 102 from the storage system 104, including the resuscitation module 72, which is an application within the software 102. The resuscitation module 72 includes computer-readable instructions that, when executed by the computing system 100 (including the processor 106), direct the processor 106 to operate as described herein.

Although the computing system 100 as depicted in FIG. 3 includes one software 102 encapsulating one resuscitation module 72, it should be understood that one or more software elements having one or more modules may provide the same operation. Similarly, while description as provided herein refers to one computing system 100 and a processor 106, it is to be recognized that the methods and systems described herein be executed using two or more computing systems (processors, storage systems, etc.), which may be communicatively connected, and such implementations (which are exemplified in the embodiment of FIG. 4) are considered to be within the scope of the description.

Processor 106 may comprise a microprocessor and other circuitry that retrieves and executes software 102 from storage system 104. Processor 106 can be implemented within a single processing device but can also be distributed across multiple processing devices or sub-systems that cooperate in executing program instructions. Examples of processor 106 include general purpose central processing units, application specific processors, and logic devices, as well as any other type of processing device, combinations of processing devices, or variations thereof.

The storage system 104 may comprise any storage media, or group of storage media, readable by processor 106 and capable of storing software 102. The storage system 104 may include volatile and non-volatile, removable and non-removable media implemented in any method or technology for storage of information, such as computer-readable instructions, data structures, program modules, or other data. Storage system 104 can be implemented as a single storage device but may also be implemented across multiple storage devices or sub-systems. Storage system 104 may further include additional elements, such a controller capable of communicating with the processor 106.

Examples of storage media include random access memory, read only memory, magnetic discs, optical discs, flash memory, virtual memory, and non-virtual memory, magnetic sets, magnetic tape, magnetic disc storage or other magnetic storage devices, or any other medium which can be used to storage the desired information and that may be accessed by an instruction execution system, as well as any combination or variation thereof, or any other type of storage medium. Likewise, the storage media may be housed locally with the processor 106, or may be distributed in one or more servers, which may be at multiple locations and networked, such as in cloud computing applications and systems. In some implementations, the storage media can be a non-transitory storage media. In some implementations, at least a portion of the storage media may be transitory.

The communication interface 108 is configured to provide communication between the processor 106 and various other systems and devices, including to receive respiration parameter measurements 90-95 from sensors 27-32 and communicate commands and information to the hub device 68 and/or host network 76. For example, communication interface 108 may control or include receiver/transmitters 35 that communicate with receiver/transmitter 34 on the respiration sensor device 26. Likewise, communication interface 108 may control or include receiver/transmitter 109 that communicates with the receiver/transmitter 209a on the hub device 68 or receiver/transmitter 309 of the host network 76. Likewise, communication interface 108 may receive information from wired connections, such as from the pulse oximeter 22 and/or ventilator device 40. Likewise, communication interface 108 may communicate with or include a controller for the digital display 46.

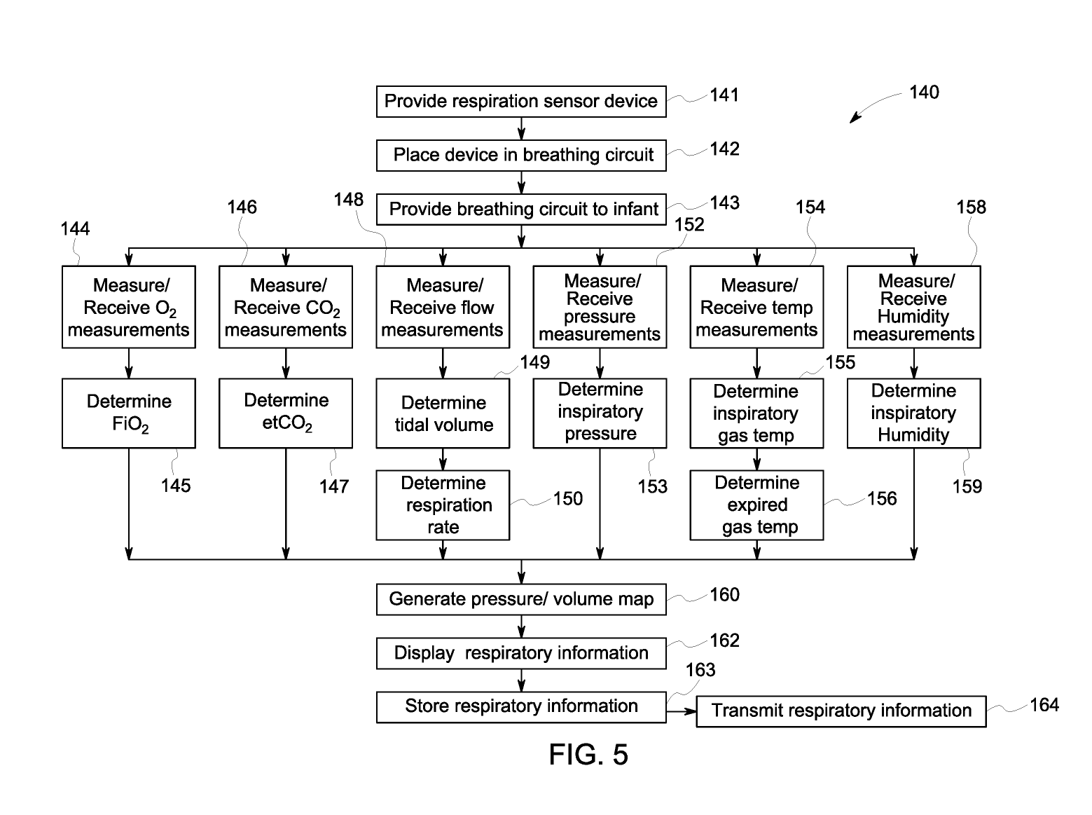

FIG. 5 depicts one embodiment of a method 140 of monitoring newborn infant respiration. A respiration sensor device 26 is provided at step 141, and the respiration sensor device 26 is placed in the breathing circuit 25 at step 142, such as between the mask 36 and breathing tube 38. The breathing circuit is provided to the infant 2 at step 143, such as by placing the mask over the infant's nose and mouth. One or more respiration parameters are measured by various sensors within the breathing circuit 25, such as O₂, CO₂, flow rate, pressure, volume, temperature, and humidity. O₂ measurements 90 are measured and/or received at step 144, such as by O₂ sensor 27, resuscitation module 72 in the software 102 of the computing system 100. The resuscitation module 72 then determines an FiO₂ value at step 145 based on the O₂ measurements 90. Similarly, CO₂ measurements 91 are measured and/or received at step 146, and an etCO₂ value is determined at step 147 based on the CO₂ measurements 91. Similarly, flow measurements 92 are measured and/or received at step 148 and a tidal volume is determined at step 149 based on the flow measurements 92. Likewise, a respiration rate may be determined at step 150 based on the flow measurements 92, such as based on the period of the flow cycle. Alternatively or additionally, the respiration rate may be determined based on different measurements, such as based on the period of the pressure cycle. Pressure measurements 93 are measured and/or received at step 152, and inspiratory pressure is determined at step 153 based thereon. Temperature measurements 94 are likewise measured and/or received at step 154, and an inspiratory gas temperature (i.e. temperature of the inspiratory gas) may be determined at step 155. For example, the inspiratory gas temperature may be the average or mean of the temperature measurements 94 recorded during the inspiratory phase of one or more breath cycles. Alternatively or additionally, an expired gas temperature is determined at step 156, such as an average or mean of the temperature measurements 94 recorded during the expiratory phase of one or more breath cycles. Humidity measurements 95 are likewise measured and/or received at step 158, and a humidity of an inspiratory gas is determined at step 159. Further respiratory information 96 may be calculated at step 160, such as comparing the inspiratory pressure and tidal volume to generate a pressure vs. volume map. Some or all of the forgoing respiratory information 96 may be displayed at step 162, such as on the digital display 46. At step 163, the respiratory information 96 is stored in memory of storage system 104. The respiratory information 96 is transmitted at step 164, such as to the hub device 68 and/or the host network 76 as described herein. In one embodiment, steps 144 through 164 are carried out by executing instructions of the resuscitation module 72 on processor 106 of the computing system 100. In another embodiment, one or more of the steps 144-159 are carried out within the respiration sensor device 26, such as by executing corresponding software instructions on a processor of first computing system 100a therein. The respective values generated at those steps may be transmitted to the second computing system 100b, which may then execute steps 162 through 164.

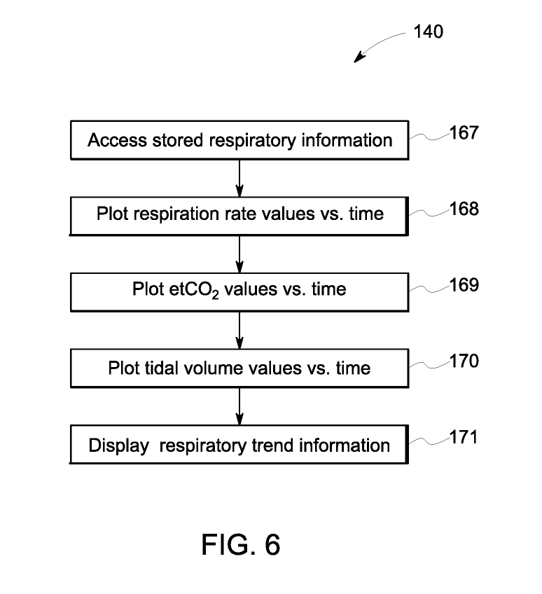

FIG. 6 depicts another embodiment of a method 140 of monitoring infant respiration where respiratory information trends are determined and displayed to assist a clinician in determining the respiratory condition or health status of the infant. At step 166, stored respiratory information 96 is accessed, such as by resuscitation module 72 within computing system 100. At step 168, all respiration rate values are plotted with respect to time, which may include all respiration rate values determined for the infant 2 since the infant's time of birth, or may include respiration rate values over a predetermined or selected period of time. Similarly, the etCO₂ values are plotted with respect to time at step 169, which may again include all values calculated since the infant's birth or a subset of those values. Likewise, tidal volume values are plotted with respect to time at step 170, which may again include all tidal volume values calculated since the infant's birth or a subset thereof. The respiratory trend information is displayed at step 171, which may include any or all of the respiration rate plot, the etCO₂ plot, and the tidal volume plot, for example.

This written description uses examples to disclose the invention, including the best mode, and also to enable any person skilled in the art to make and use the invention. Certain terms have been used for brevity, clarity and understanding. No unnecessary limitations are to be inferred therefrom beyond the requirement of the prior art because such terms are used for descriptive purposes only and are intended to be broadly construed. The patentable scope of the invention is defined by the claims, and may include other examples that occur to those skilled in the art. Such other examples are intended to be within the scope of the claims if they have features or structural elements that do not differ from the literal language of the claims, or if they include equivalent features or structural elements with insubstantial differences from the literal languages of the claims.

We claim:

1. A newborn respiration monitoring system comprising:
a flow sensor that measures a gas flow within a breathing circuit for an infant;
a CO₂ sensor that measures a CO₂ within the breathing circuit for the infant;
a resuscitation module executable on a processor of a computing system to receive the flow measurement and the CO₂ measurement and determine respiratory information for the infant; and
a digital display communicatively connected to the computing system that displays the respiratory information.

2. The newborn respiration monitoring system of claim 1, wherein the respiratory information includes a tidal volume and an end tidal CO₂ (etCO₂).

3. The newborn respiration monitoring system of claim 1, further comprising a respiration sensor device in the breathing circuit containing the CO₂ sensor and the flow sensor.

4. The newborn respiration monitoring system of claim 3, wherein the respiration sensor device is positionable between a mask and a breathing tube of the breathing circuit.

5. The newborn respiration monitoring system of claim 4, wherein the respiration sensor device has a first end removably connectable to the mask and a second end removably connectable to the breathing tube.

6. The newborn respiration monitoring system of claim 3, further comprising an O₂ sensor in the respiration sensor device that measures O₂ within the breathing circuit for the infant, wherein the respiratory information determined by the resuscitation module further includes fraction of inspired oxygen (FiO₂).

7. The newborn respiration monitoring system of claim 3, further comprising a pressure sensor in the respiration sensor device that measures pressure within the breathing circuit for the infant, wherein the respiratory information determined by the resuscitation module further includes an inspiratory pressure.

8. The newborn respiration monitoring system of claim 3, wherein the respiration sensor device contains the processor and at least a portion of the resuscitation module.

9. The newborn respiration monitoring system of claim 8, wherein the respiration sensor device wirelessly transmits the respiratory information to the computing system.

10. The newborn respiration monitoring system of claim 1, further comprising at least one of an O₂ sensor that measures O₂ within the breathing circuit, a pressure sensor that measures pressure within the breathing circuit, a temperature sensor that measures temperature within the breathing circuit, and a humidity sensor that measures humidity within the breathing circuit, wherein the respiratory information determined by the resuscitation module further includes at least one of a fraction of inspired oxygen (FiO₂), an inspiratory pressure, an inspiratory gas temperature, an expired gas temperature, an inspiratory gas humidity, and a respiration rate.

11. The newborn respiration monitoring system of claim 1, further comprising at least one of a pulse oximeter device and a co-oximeter device configured to determine at least one of an SpO₂, a SpHb, an SpMet, and an SpCO for the infant, and wherein the resuscitation module is executable to display the at least one of the SpO₂, the SpHb, the SpMet, and the SpCO on the digital display.

12. The newborn respiration monitoring system of claim 1, wherein the resuscitation module is further executable to effect transmission of the respiratory information to at least one of a hub device or a host network.

13. The newborn respiration monitoring system of claim 1, wherein the resuscitation module is further executable to determine one or more respiratory information trends for the infant over a period of time, and to display the one or more respiratory information trends on the digital display.

14. The newborn respiration monitoring system of claim 1, further comprising a mask in the breathing circuit containing the CO₂ sensor and the flow sensor.

15. A method of monitoring newborn infant respiration, the method comprising:
measuring a gas flow with a flow sensor in a breathing circuit for the infant;
communicating the flow measurement to a computing system;
measuring a CO₂ with a CO₂ sensor in the breathing circuit for the infant;
communicating the CO₂ measurement to the computing system;
determining respiratory information for the infant with the computing system based on at least the flow measurement and the CO₂ measurement; and
displaying the respiratory information for the infant on a digital display communicatively connected to the computing system.

16. The method of claim 15, wherein the wherein the respiratory information includes at least one of a tidal volume, an end tidal CO₂ (etCO₂), and a respiration rate.

17. The method of claim 15, further comprising at least one of measuring an O₂ within the breathing circuit with an O₂ sensor, measuring a pressure of inspired gas within the breathing circuit with a pressure sensor, measuring a temperature within the breathing circuit with a temperature sensor, and measuring a humidity within the breathing circuit with a humidity sensor, and wherein the step of determining respiratory information further includes determining at least one of a fraction of inspired oxygen (FiO₂), an inspiratory pressure, an inspiratory gas temperature, an expired gas temperature, an inspiratory gas humidity, and a respiration rate for the infant.

18. The method of claim 17, further comprising generating a pressure volume map for the infant based on the pressure measurement and the tidal volume.

19. The method of claim 15, further comprising generating a respiratory information trend for the infant over a period of time and displaying the respiratory information trend on the digital display.

20. The method of claim 15, further comprising supplying a respiration sensor device communicatively connected to the computing system, wherein the respiration sensor device contains at least the flow sensor and the CO₂ sensor and is connectable within the breathing circuit for the infant.
